(19) United States
(12) Patent Application Publication
(10) Pub. No.: US 2024/0230635 A1
(43) Pub. Date: Jul. 11, 2024

(54) METHOD FOR PERFORMING SIMPLIFIED ELISA OPERATION

(71) Applicant: MULTI SCIENCES(LIANKE) BIOTECH CO.,LTD., Hangzhou (CN)

(72) Inventors: LUHONG SONG, Hangzhou (CN); SHUAI WANG, Hangzhou (CN); HANGNA ZHAO, Hangzhou (CN)

(21) Appl. No.: 18/369,340

(22) Filed: Sep. 18, 2023

(30) Foreign Application Priority Data
Jan. 8, 2023 (CN) 202310022937.8

(51) Int. Cl. G01N 33/543; G01N 33/96

(52) U.S. Cl. CPC G01N 33/54386; G01N 33/96; G01N 2496/80

(57) ABSTRACT

The present invention discloses a method for performing enzyme linked immunosorbent assay (ELISA) with simplified operation, including: preparing standards of a target protein with gradient dilutions and pre-diluting test samples; setting up wells for the standards and wells for the test samples on an ELISA plate, adding 100 μl of the diluted standards and 100 μl of the diluted samples into their respective wells, incubating, discarding the liquid, drying, washing the residue; adding 100 μl of TMB chromogenic substrate into the wells of the standards and the wells of the test samples respectively, incubating at room temperature, adding 100 μl of a stop solution to terminate the reaction in each well; placing the ELISA plate into an ELISA reader for dual-wavelength detection, calculating the concentration of a target protein in the samples. The overall operation time can be controlled within 90 minutes at minimum.

### CROSS-REFERENCE TO RELATED APPLICATIONS

This is a U.S. patent application which claims the priority and benefit of Chinese Patent Application Number 202310022937.8, filed on Jan. 8, 2023, the disclosure of which is incorporated herein by reference in its entirety.

### TECHNICAL FIELD

The present invention belongs to the field of enzyme-linked immunosorbent assay (ELISA) experimental techniques, and particularly relates to an ELISA plate to perform simplified ELISA operation and the application thereof.

### BACKGROUND

Enzyme-linked immunosorbent assay (ELISA) is a classic immunological detection method that can qualitatively and quantitatively detect target substances. It is characterized by high sensitivity, high specificity, and good repeatability.

Based on differences in operational steps and target substances to be detected, ELISA experiments can be classified into various types, such as direct method, indirect method, double antibody sandwich method, and competitive method. Among them, the most commonly used is double antibody sandwich method. In brief this commonly used method includes the following steps: pre-coating the capturing antibodies on the ELISA plate, adding standards/test samples, adding detecting antibodies, adding horseradish peroxidase (HRP) enzyme, adding TMB (3,3′,5,5′-tetramethylbenzidine) chromogenic substrate, adding stop solution, and reading assay result with ELISA reader, and so on. Each of those steps before adding TMB chromogenic substrate is followed with plural steps of washing. Consequently, traditional ELISA experiments are cumbersome, time-consuming, and prone to human errors. Additionally, the detecting antibodies can be biotinylated and conjugated with HRP enzyme, which may skip the step of adding HRP enzyme but does not significantly reduce the overall experiment time or eliminate the aforementioned drawbacks.

### SUMMARY

The main objective of this application is to provide a method to solve the problems like cumbersome and time-consuming steps in existing ELISA operation by designing a simplified sandwich ELISA method, which reduces the number of steps and shortens the overall operation time.

To achieve the above objective, the present invention provides the following technical solutions:

A method for performing simplified ELISA operation, comprising the following steps:
S1: Preparing standards of a target protein with gradient dilutions, and pre-diluting test samples;
S2: Setting up wells for the standards and wells for the test samples on an ELISA plate, adding 100 μl of diluted standards/test samples into their respective wells, incubating, discarding the liquid, washing after drying;
S3: Adding 100 μl of TMB chromogenic substrate into each well of standards/test samples, incubating at room temperature, adding 100 μl of stop solution into each well to terminate the reaction;
S4: Placing the ELISA plate into an ELISA reader for dual-wavelength detection, reading the results of the standards, plotting OD curve of the standards, and calculating the concentration of the target substance in the samples.

In the method for performing simplified ELISA operation described in this application, the detecting antibodies and/or HRP enzyme are used to prepare homogeneous microspheres and preplaced in a 96-well plate. That is to say, the steps before TMB chromogenic step in the traditional ELISA method are integrated as one step, and the washing step is only required once after each time of incubating antibodies. The minimum overall operation time is controlled within 90 minutes at minimum.

This method simplifies and streamlines ELISA operation, greatly reducing the overall experiment time. Additionally, the reduction in the number of steps lowers the chances of operation errors and improves detection repeatability of the assay kit.

In one preferred embodiment of the method for performing simplified ELISA operation, in S1, the specific operation for gradient dilution is performed at 7 different concentration dilutions as required, using a dilution solution of phosphate-buffered saline (PBS) containing 0.5% bovine serum albumin (BSA).

In one preferred embodiment of the method for performing simplified ELISA operation, in S2, the incubating condition is incubating at 37° C. for 1 hour, and the washing solution as used is 1×PBS-T solution.

In one preferred embodiment of the method for performing simplified ELISA operation, in S3, the incubating condition is at room temperature for 5-15 minutes, and the stop solution is a sulfuric acid solution with a concentration of 1 M.

In one preferred embodiment of the method for performing simplified ELISA operation, in S4, the main wavelength for dual-wavelength detection is 450 nm, and the reference wavelength is 570 nm or 630 nm.

In one preferred embodiment of the method for performing simplified ELISA operation, the manufacturing method of ELISA plate comprises the following steps:
Step 1: Diluting capturing antibodies of a target protein to a working concentration by using PBS buffer, adding 50 μl per well to a 96-well plate, and incubating overnight at 4° C. for coating;
Step 2: Discarding the excess liquid, tap-drying, and adding 300 μl of 5% BSA solution into each well, keeping it at room temperature for 2 hours, wherein, BSA solution is bovine serum albumin (BSA), a protective membrane layer that can be solved into water later so as to expose capturing antibodies is formed thereupon;
Step 3: Discarding the liquid, tap-drying, adding 300 μl of a solution containing sucrose, trehalose, and gelatin into each well, placing at room temperature for 15 minutes, discarding the excess liquid, tap-drying and then heat-drying;
Step 4: Diluting detecting antibodies of a target protein to a working concentration by using an antibody stabilizer, forming antibody microspheres; and preparing HRP enzyme microspheres;
Step 5: Placing the microspheres obtained in Step 4 into the heat-dried 96-well plate obtained in Step 3, so as to prepare the ELISA plate.

In one preferred embodiment of the method for performing simplified ELISA operation, in step 1, the working concentration is 10 ng-20 μg/ml.

In one preferred embodiment of the method for performing simplified ELISA operation, in step 3, the mass concentration of sucrose in water solution is 30%, the mass concentration of trehalose is 10%, and the mass concentration of gelatin is 5%.

In one preferred embodiment of the method for performing simplified ELISA operation, in step 3, the drying temperature is 20° C., and the drying time is 2 hours.

In one preferred embodiment of the method for performing simplified ELISA operation, in step 4, the antibody stabilizer is BioStab antibody stabilizer, with a working concentration of 1 ng/ml-100 ng/ml, the particle size of the antibody microspheres is 1-5 mm, and the particle size of the HRP enzyme microspheres is 1-5 mm.

Additionally, the invention also provides an ELISA plate to perform simplified operation, which includes a plate body and a sealing membrane layer covering the plate body. Inside the wells of the plate body is covered with a composite layer, which includes, from bottom to top, a capturing antibody layer for target protein, a protein protection layer, and a gelatin protective membrane layer.

The microspheres comprising detecting antibodies and HRP enzyme are contained within the 96 wells. The microspheres can be detecting antibody microspheres and HRP enzyme microspheres. Similarly, the microspheres can also be conjugated microspheres of detecting antibodies and HRP enzymes.

That is to say, there is one kind of antibody microsphere in the wells of the plate body, or there are two kinds of microspheres including antibody microspheres and HRP enzyme microspheres, or there is one kind of microspheres prepared by conjugating detecting antibodies with HRP enzyme.

The positions of the sealing membrane corresponding to the wells are all provided with liquid injection openings, which formed as small slits in the shape of a straight line, a cross, or a double cross, for adding reagents in later detecting process.

The beneficial effects of the present invention are as follows: with the method for performing simple ELISA operation as described in the present invention, the detecting antibodies and/or HRP enzyme are used to prepare uniform microspheres and pre-placed in the ELISA plate, and the operation before TMB color development can be integrated as one step, and it only needs to be washed once after each time of antibody incubation, and the minimum overall operation time can be controlled within 90 minutes.

This method simplifies the ELISA operation and makes it more convenient, therefore, it can greatly reduce the overall experiment time. In addition, due to the reduction of the number of operating steps, the probability of operating errors can be decreased, and the detection repeatability of the assay kit can be increased. In summary, the method of the present invention is simple to operate, it saves time and efforts, the overall process is controllable, and the products thereof can be well-functional.

### BRIEF DESCRIPTION OF DRAWINGS

FIG. 1 is the ELISA detection result of the human PDGFR protein standards detected by the method of the present invention, in Example 1 of the present invention;

FIG. 2 is the ELISA detection result of the human IFN-γ protein standards detected by the method of the present invention, in Example 3 of the present invention.

### DETAILED DESCRIPTION

To be better understood by those skilled in the art, the technical solution in the examples of this application, will be described clearly and thoroughly with specific case. Apparently, it should be noted that the examples described herein are only part of the embodiment of the present application, and not the entire embodiment. All other embodiment obtained based on the examples without inventive efforts by ordinary skilled persons in the art in this application should also be within the protection scope of this application.

With the detection method of the present application, different types of substances, such as serum, plasma, cell culture supernatant, can be detected. Different pretreatment like diluting, condensing, and etc may be necessary for special samples due to different concentrations of the target substance in different samples.

The microspheres described in the present application can be prepared in different shapes, including but not limited to, sphere, ellipse, and irregular shapes.

### Example 1

The capturing antibodies for anti-human PDGFR protein is pre-coated at the bottom of the ELISA plate. HRP enzyme and anti-human PDGFR protein are formed into microspheres with a diameter of 3 mm and pre-placed in the ELISA plate. The plate is vacuum-scaled in an aluminum foil bag for storage.

The specific operation method for preparing ELISA plate is as follows:

Diluting the capturing antibodies for anti-human PDGFR protein to a concentration of 10 ug/ml using PBS buffer, adding 50 μl per well to a 96-well plate, and keeping at 4° C. overnight for coating (the coating forms a a capturing antibody layer on the 96-well plate). Discarding the excess liquid, drying the plate by tapping the bottom to completely remove the excess liquid, so as to form a capturing antibody layer.

Adding 300 μl of 5% BSA solution to each well, keeping at room temperature for 2 hours (forming a protein protection layer on the surface of the coated a capturing antibody layer to prevent other proteins from coming into contact with capturing antibodies). Then, discarding the liquid, tap-drying, so as to form a protein protection layer.

Adding 300 μl of water solution containing sucrose, trehalose, and gelatin to each well. The mass concentration of sucrose in water solution is 30%, the mass concentration of trehalose is 10%, and the mass concentration of gelatin is 5%. Keeping at room temperature for 15 minutes to form a protective membrane layer. Discarding the excess liquid, drying the plate by tapping, and then drying it at 20° C. for 2 hours, so as to form a gelatin protective membrane layer.

Diluting the detecting antibodies for anti-human PDGFR protein to a concentration of 10 ng/ml using an antibody stabilizer (BioStab antibody stabilizer), so as to form antibody microspheres with a diameter of 3 mm, and preparing HRP enzyme microspheres with a diameter of 3 mm.

Placing antibody microspheres and HRP enzyme microspheres into the heat-dried 96-well plate obtained in the previous step, so as to prepare the ELISA plate. Placing the ELISA plate in an aluminum foil bag, vacuum-sealing, and storing it at 2-8° C. for later use.

The method for detecting the concentration of human PDGFR protein by using the method described in the present invention is as follows:
(1) Taking out the sealed ELISA plate from the 2-8° C. environment in advance, letting it equilibrate at room temperature for at least 15 minutes, so as to make the plate ready for use.
(2) Preparing 7 gradient dilutions of human PDGFR protein standards (specific concentrations shown in the first column of Table 1) and diluting test samples by 10 times.
(3) Setting up wells for the standards/test samples on the ELISA plate, adding 100 μl of 7 different concentrations of standards/test samples into their respective wells, incubating at 37° C. for 1 hour, discarding the liquid, and tap-drying, washing the wells with 1×PBS-T solution for 3-6 times.
(4) Adding 100 μl of TMB chromogenic substrate to each well of the standards and the test samples, incubating at room temperature for 10 minutes, then adding 100 μl of 1 M sulfuric acid as the stop solution into each well.
(5) Placing the ELISA plate into an ELISA reader for dual-wavelength detection, wherein, the main wavelength is 450 nm, and the reference wavelength is 570 nm or 630 nm, plotting OD curve of the standards based on the reading results of the standards, and calculating the concentration of human PDGFR protein in the samples.

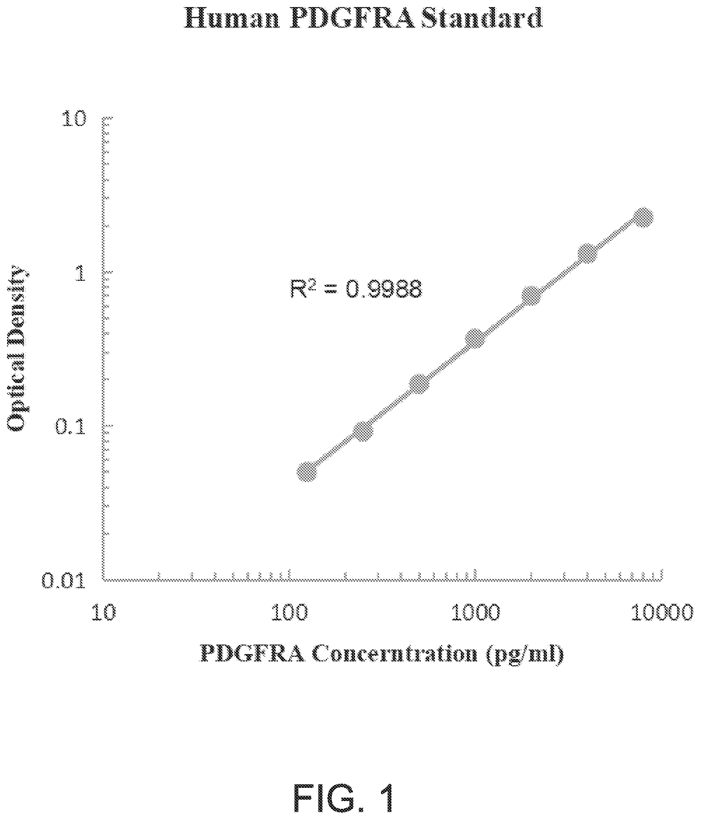

The OD values of the standard curve in Example 1 are shown in Table 1, and the corresponding standard curve is shown in FIG. 1.

TABLE 1

| Concentration of 7 | OD | OD | Corrected |
|---|---|---|---|
| gradient levels (pg/ml) | value1 | value2 | OD value |
| 1000 | 2.558 | 2.565 | 2.517 |
| 500 | 1.381 | 1.330 | 1.311 |
| 250 | 0.658 | 0.651 | 0.610 |
| 125 | 0.363 | 0.374 | 0.324 |
| 62.5 | 0.195 | 0.196 | 0.151 |
| 31.25 | 0.147 | 0.152 | 0.105 |
| 15.625 | 0.078 | 0.085 | 0.037 |
| 0 | 0.042 | 0.047 | 0.000 |

From Table 1, it can be seen that the OD values of the standard curve in this embodiment form a proportional gradient. The background value (at 0 concentration) is less than 0.1, and the corresponding standard curve fitting coefficient R2 is greater than 0.99. The overall performance of the standard curve drawn by using the method of the present invention is excellent.

### Example 2

In this embodiment, anti-mouse TNF-α protein was conjugated with HRP enzyme to form microspheres, which were then placed in an ELISA plate coated with capturing antibodies of anti-mouse TNF-α protein.

The specific operational method for preparing the ELISA plate is as follows:

Diluting the capturing antibodies of anti-mouse TNF-α protein to a concentration of 20 μg/ml by using PBS buffer solution, adding 50 μl per well to a 96-well plate, and keeping it overnight at 4° C. for coating (coating refers to the formation of a layer of capturing antibodies membrane in the 96-well plate). Discarding the excess liquid and tap-drying (tapping the bottom of the 96-well plate to completely remove the excess liquid). Then adding 300 μl of 5% BSA solution to each well, keeping it at room temperature for 2 hours (forming a protein protective membrane layer on the surface of the capturing antibodies to prevent capturing antibodies from contacting other proteins), then discarding the liquid and tap-drying.

Adding 300 μl of water solution containing sucrose, trehalose, and gelatin to each well, wherein, the mass concentration of sucrose in water solution is 30%, trehalose is 10%, and gelatin is 5%, keeping it at room temperature for 15 minutes to form a protective membrane layer, discarding the excess liquid and tap-drying, then keeping drying it at 20° C. for 2 hours.

Diluting the detecting antibodies of anti-mouse TNF-α protein (previously conjugated with HRP enzyme) to a concentration of 10 ng/ml by using BioStab antibody stabilizer (purchased from Bitop, Germany), so as to prepare microspheres with a diameter of 3 mm.

Placing the microspheres into the heat-dried 96-well plate, so as to form ELISA plate. Placing the ELISA plate in an aluminum foil bag, vacuum-sealing it, and storing it at 2-8° C. for future use.

The detection method for measuring the concentration of anti-mouse TNF-α protein by using the method described in the present invention is as follows:
1. Taking out the sealed ELISA plate from the 2-8° C. environment in advance, letting it equilibrate at room temperature for at least 15 minutes, so as to prepare the plate ready for use.
2. Preparing diluted standards of mouse TNF-α protein with 7 gradient (as shown in Table 2) and pre-diluting the test samples of mouse serum.
3. Setting up wells for the standards and wells for the test samples on the ELISA plate. Adding 100 μl of the diluted standards/test samples of 7 concentrations into the respective wells. Incubating it at 37° C. for 1 hour, discarding the liquid, tap-drying, and washing with 1×PBS-T solution for 3-6 times.
4. Adding 100 μl of TMB chromogenic substrate into the wells for the standards and the welss for the test samples, incubating it at room temperature for 5-15 minutes, then adding 100 μl of 1 M sulfuric acid stop solution into each well.
5. Placing the ELISA plate into an ELISA reader for dual-wavelength detection, wherein, the main wavelength is 450 nm, and the reference wavelength is 570 nm or 630 nm, plotting OD curve of the standards based on the reading results of the standards, and calculating the concentration of mouse TNF-α protein in the samples.

Simultaneously, using ELISA plate and detecting antibodies which are not processed with the method of the present invention, by using the traditional method, which involves multiple operation and washing steps, to measure the 7 gradient-diluted standards of mouse TNF-α protein and the test samples of mouse serum as in the above steps.

The traditional detection method is as follows:
Step 1. Setting up wells for the standards, wells for the blank and the wells for the test samples respectively, more specifically, adding 100 μL of diluted standards to the standard wells, adding 100 μL of diluent of standards/test samples to the blank wells, and adding 100 μL of the samples in the remaining wells, so as to form a coating membrane layer for the ELISA plate, and incubating it at 37° C. for 90 minutes;
Step 2. Shaking off the liquid in the wells without washing, adding 100 μL of biotinylated antibody working solution into each well, so as to form a coating membrane layer for the ELISA plate, and incubating it at 37° C. for 1 hour,
Step 3. Shaking off the liquid in the wells and tap-drying on clean absorbent paper, adding 350 μL of washing solution into each well, soaking for 1 minute, absorbing or shaking off the liquid in the ELISA plate, and tap-drying, and then repeating the washing step for 3 times, proceeding to the next step immediately after washing the plate, before the ELISA plate become dry;
Step 4. Adding 100 μL of enzyme-conjugated working solution into each well, so as to form a coating membrane layer onto the ELISA plate, and incubating it at 37° C. for 30 minutes;
Step 5. Shaking off the liquid in the wells, washing the plate for 5 times, the method is the same as in Step 3;
Step 6. Adding 90 μL of substrate solution (TMB) into each well, so as to form a coating membrane layer on the ELISA plate, and incubating it at 37° C. in the dark for about 15 minutes.
Step 7. Adding 50 μL of stop solution to each well to stop the reaction. Tip: The adding order of the stop solution should be the same as that of the substrate solution.
Step 8. Immediately measuring the optical density (OD value) of each well with an ELISA plate reader at a wavelength of 450 nm.

The OD values of the standard curve obtained by both methods are shown in Table 2.

TABLE 2

| | | | | The method of the | | |
|---|---|---|---|---|---|---|
| | Traditional operation method | | | present invention | | |
| Concentration | OD | OD | Relative | OD | OD | Relative |
| of 7 gradient | value | value | biased | value | value | biased |
| levels (pg/ml) | 1 | 2 | CV value | 1 | 2 | CV value |
| 700 | 2.368 | 2.233 | 4.15% | 2.416 | 2.395 | 0.62% |
| 350 | 1.253 | 1.141 | 6.62% | 1.529 | 1.480 | 2.30% |
| 175 | 0.533 | 0.569 | 4.62% | 0.926 | 0.883 | 3.36% |
| 87.5 | 0.282 | 0.248 | 9.07% | 0.514 | 0.487 | 3.81% |
| 43.75 | 0.131 | 0.143 | 6.19% | 0.316 | 0.321 | 1.11% |
| 21.875 | 0.094 | 0.105 | 7.82% | 0.219 | 0.210 | 2.97% |
| 10.9375 | 0.051 | 0.046 | 7.29% | 0.174 | 0.168 | 2.48% |
| 0 | 0.030 | 0.027 | 7.44% | 0.051 | 0.049 | 2.83% |
| Average value | / | / | 6.65% | / | / | 2.44% |

From Table 2, it can be seen that the OD values of the standard curves for both methods exhibit a proportional gradient. The background value (at 0 concentration) is below 0.1, and the corresponding standard curve fitting coefficient R2 is greater than 0.99. Additionally, the average coefficient of variation (CV) of the duplicate wells in the standard curve based on the method of the present invention is smaller than that of the traditional method, indicating that the repeatability of the results obtained by using the method of the present invention is better.

In normal mouse serum, the concentration of TNF-α is typically low or undetectable. If necessary, samples from animals in an inflammation model experiment can be used.

### Example 3

In this example, the capturing antibodies of anti-human IFN-γ protein was coated at the bottom of the ELISA plate. Biotinylated detecting antibodies and HRP enzyme were formed as microspheres and pre-placed in the ELISA plate, and then vacuum-sealed in an aluminum foil bag for storage.

The specific procedure for preparing the ELISA plate is as follows:

Diluting the capturing antibodies of anti-human IFN-γ protein to a concentration of 10 μg/ml by using PBS buffer solution. More specifically, adding 50 μl per well into a 96-well plate, keeping it overnight at 4° C. for coating (coating refers to the formation of a capturing antibodies membrane layer onto the 96-well plate). Discarding the excess liquid, and tap-drying, then adding 300 μl of 5% BSA solution into each well and keeping it at room temperature for 2 hours (to form a protein protective membrane layer onto the surface of the coated capturing antibodies membrane, so as to prevent other proteins from contacting the capturing antibodies). Then discarding the liquid, tap-drying. Next, adding 300 μl of a water solution containing sucrose, trehalose, and gelatin into each well, wherein, the mass concentration of sucrose in water solution is 30%, trehalose is 10%, and gelatin is 5%. Keeping it at room temperature for 15 minutes to form a protective membrane layer, then discarding the excess liquid and tap-drying, and then keeping drying it at 20° C. for 2 hours.

Diluting the detecting antibodies of anti-human IFN-γ protein (previously conjugated with HRP enzyme) to a concentration of 50 ng/ml by using BioStab antibody stabilizer (purchased from Bitop, Germany), so as to form microspheres with a diameter of 3 mm.

Placing the microspheres into the heat-dried 96-well plate, so as to form ELISA plate. Placing the ELISA plate in an aluminum foil bag, vacuum-sealing it, and storing it at 2-8° C. for future use.

The detection method for measuring the concentration of human IFN-γ protein using the method described in the present invention is as follows:
1. Taking out the sealed ELISA plate from the 2-8° C. environment in advance, letting it equilibrate at room temperature for at least 15 minutes, and unsealing it.
2. Preparing diluted standards of human IFN-γ protein with 7 gradient (as shown in Table 3) and pre-diluting the samples of human IFN-γ protein.
3. Setting up wells for the standards and wells for the test samples on the ELISA plate, adding 100 μl of the diluted standards/test samples of 7 concentrations into the respective wells. Incubating it at 37° C. for 1 hour, discarding the liquid, tap-drying, and washing with 1×PBS-T solution for 3-6 times.
4. Adding 100 μl of TMB chromogenic substrate into the wells for stands/samples, incubating it at room temperature for 5-15 minutes, and then adding 100 μl of 1 M sulfuric acid stop solution into each well.
5. Placing the ELISA plate into an ELISA reader for dual-wavelength detection, wherein, the main wavelength is 450 nm, and the reference wavelength is 570 nm or 630 nm, plotting OD curve of the standards based on the reading results of the standards, and calculating the concentration of mouse TNF-α protein in the samples.

The detecting antibodies of anti-human IFN-γ protein (previously conjugated with HRP enzyme) was diluted to a concentration of 50 ng/ml using BioStab antibody stabilizer (purchased from Bitop, Germany) and formed as microspheres with a diameter of 3 mm.

The microspheres were placed into the dried 96-well plate, resulting in the ELISA plate. The ELISA plate was then placed in an aluminum foil bag, vacuum-sealed, and stored at 2-8° C. for later use.

The method for detecting the concentration of human IFN-γ protein by using the method of the present invention is as follows:
1. Taking out the sealed ELISA plate from the 2-8° C. environment in advance, letting it to equilibrate at room temperature for 15 minutes, and then preparing the ELISA plate ready for use.
2. Preparing 7 gradient diluted human IFN-γ protein standards (as shown in Table 3) and pre-diluting tested samples of human IFN-γ protein.
3. Setting up wells for the standards and wells for the test samples on the ELISA plate, adding 100 μl of the diluted standards/test samples of 7 concentrations into the respective wells, incubating it at 37° C. for 1 hour, discarding the liquid, tap-drying, and washing with 1×PBS-T solution for 3-6 times.
4. Adding 100 μl of TMB chromogenic substrate into the standard wells and sample wells, incubating it at room temperature for 5-15 minutes, and then adding 100 μl of 1 M sulfuric acid stop solution into each well.
5. Placing the ELISA plate into an ELISA reader for dual-wavelength detection, wherein, the main wavelength is 450 nm, and the reference wavelength is 570 nm or 630 nm. Based on the reading results of the standards, plotting OD curve of the standards and calculating the concentration of human IFN-γ protein in the samples.

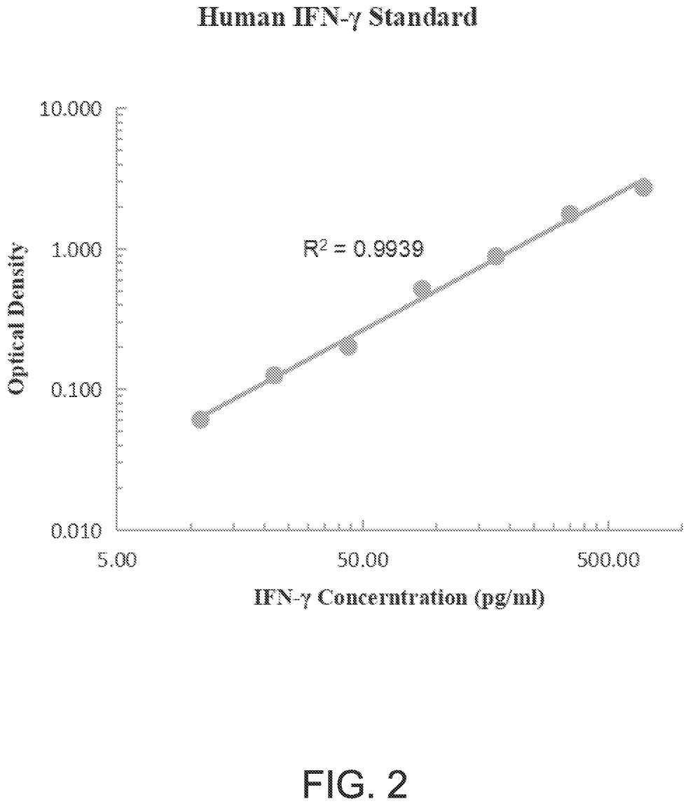

The OD values of the standard curve are shown in Table 3, and the corresponding standard curve graph is shown in FIG. 2.

TABLE 3

| Concentration of 7 | OD | OD | Corrected |
|---|---|---|---|
| gradient levels (pg/ml) | value1 | value2 | OD value |
| 700 | 2.819 | 2.807 | 2.728 |
| 350 | 1.814 | 1.900 | 1.772 |
| 175 | 1.017 | 0.923 | 0.885 |
| 87.5 | 0.619 | 0.588 | 0.519 |
| 43.75 | 0.293 | 0.281 | 0.202 |
| 21.875 | 0.215 | 0.207 | 0.126 |
| 10.938 | 0.139 | 0.153 | 0.061 |
| 0 | 0.081 | 0.089 | 0.000 |

From Table 3, it can be seen that the OD values of the standard curve exhibit a proportional gradient. The background value (at 0 concentration) is below 0.1, and the corresponding standard curve fitting coefficient R2 is greater than 0.99. The results obtained by using this method comply with the standards for practical detection.

The above description is only concerning one preferred embodiment of the present invention. It should be noted that ordinary skilled persons in the technical field can make various improvements and additions without departing from the scope of the present invention as long as they do not deviate from the method of the present invention.

What is claimed is:

1. A method for performing simplified ELISA operation, characterized by comprising the following steps:
S1, preparing standards of a target protein with gradient dilutions, and pre-diluting test samples;
S2, setting up wells for standards and wells for test samples on an ELISA plate, adding 100 μl of diluted standards and 100 μl of test samples into their respective wells, incubating, discarding the liquid, drying, and washing;
S3, adding 100 μl of TMB chromogenic substrate into each well of the standards/test samples, incubating at room temperature, adding 100 μl of stop solution to terminate the reaction in each well;
S4, placing the ELISA plate into an ELISA reader for dual-wavelength detection, reading test results based on the standards, plotting OD curve of the standards, and calculating the concentration of the target substance in the samples.

2. The method for performing simplified ELISA operation according to claim 1, wherein in S1, the gradient dilutions comprise dilutions of 7 different concentrations.

3. The method for performing simplified ELISA operation according to claim 1, wherein in S2, the incubating is under the condition of incubating temperature at 37° C., for 1 hour, and 1×PBS-T solution is used as washing solution during the washing.

4. The method for performing simplified ELISA operation according to claim 1, wherein in S3, the incubating is under the condition of incubating temperature at room temperature, for 5-15 minutes; and the stop solution is a sulfuric acid solution with a concentration of 1 M.

5. The method for performing simplified ELISA operation according to claim 1, wherein in S4, the main wavelength for dual-wavelength detection is 450 nm, and the reference wavelength is 570 nm or 630 nm.

6. The method for performing simplified ELISA operation according to claim 1, the preparing method of the ELISA plate comprising the following steps:
Step 1, diluting the capturing antibodies of the target protein to a working concentration by using PBS buffer, adding 50 μl of the PBS buffer per well into a 96-well plate, and incubating overnight at 4° C. so as to coat;
Step 2, discarding the excess liquid, drying, and adding 300 μl of 5% BSA solution to each well, placing at room temperature for 2 hours;
Step 3, discarding the liquid, drying, adding 300 μl of the water solution containing sucrose, trehalose, and gelatin to each well, placing at room temperature for 15 minutes, discarding the excess liquid, tap-drying;
Step 4, diluting detecting antibodies of a target protein to a working concentration using an antibody stabilizer, so as to form antibody microspheres; and forming HRP enzyme microspheres with HRP enzyme;
Step 5, placing the microspheres obtained in Step 4 into heat-dried 96-well plate obtained in Step 3, so as to obtain the ELISA plate.

7. The method for performing simplified ELISA operation according to claim 6, wherein in Step 1, the working concentration is 10-20 μg/ml.

8. The method for performing simplified ELISA operation according to claim 6, wherein in Step 3, the mass concentration of sucrose in the water solution is 30%, the mass concentration of trehalose is 10%, and the mass concentration of gelatin is 5%.

9. The method for performing simplified ELISA operation according to claim 6, wherein in Step 3, the drying is under the temperature of 20° C., for 2 hours.

10. The method for performing simplified ELISA operation according to claim 6, wherein in Step 4, the antibody stabilizer is BioStab antibody stabilizer, with a working concentration of 1-100 ng/ml; the particle size of the antibody microspheres is 1-5 mm, and the particle size of the HRP enzyme microspheres is 1-5 mm.
